(19) United States
(12) Patent Application Publication
(10) Pub. No.: US 2025/0361142 A1
(43) Pub. Date: Nov. 27, 2025

(54) GAS SENSOR AND METHOD FOR MANUFACTURING THEREOF

(71) Applicant: Infineon Technologies AG, Neubiberg (DE)

(72) Inventors: Vladislav KOMENKO, Dresden (DE); Albrecht HOPFE, Dresden (DE); Andrey KRAVCHENKO, Dresden (DE); Alexander KAUFMANN, Freiberg (DE); Heiko FRÖHLICH, Radebeul (DE); Thoralf KAUTZSCH, Dresden (DE); Matthias EBERL, Taufkirchen (DE); Nahas Hassan ANNACOT, Dresden (DE)

(21) Appl. No.: 19/207,962

(22) Filed: May 14, 2025

(30) Foreign Application Priority Data
May 24, 2024 (DE) 102024204805.4

(51) Int. Cl. B81B 7/00; B81C 1/00; G01N 33/00

(52) U.S. Cl. CPC B81B 7/0061; B81C 1/00309; G01N 33/0014; B81B 2201/0214; B81B 2203/0127; B81B 2203/0315; B81B 2203/0353; B81C 2201/013

(57) ABSTRACT

A gas sensor includes a microelectromechanical systems (MEMS) sensing element, a first cavity arranged in the gas sensor, and a first membrane substantially permeable for molecules of an analysis gas and substantially impermeable for molecules larger than molecules of the analysis gas. The first membrane is configured to allow a diffusion of the analysis gas into the first cavity. The MEMS sensing element is sensitive with respect to the analysis gas diffused into the first cavity.

### CROSS-REFERENCE TO RELATED APPLICATION

This application claims priority to Germany Patent Application No. 102024204805.4 filed on May 24, 2024, the content of which is incorporated by reference herein in its entirety.

### TECHNICAL FIELD

The present disclosure relates to gas sensors and methods for manufacturing gas sensors.

### BACKGROUND

Gas sensors, such as hydrogen sensors, can be used, for example, in the automotive sector or in a variety of industrial applications. Measurement approaches of gas sensors may be based on a chemical reaction (e.g., metal oxide (MOX) sensors) or a change in a physical property (e.g., thermal conductivity sensors). Although physical sensors may be more reliable, they can be subject to crosstalk. Manufacturers and developers of gas sensors are constantly striving to improve their products. In this context, it may be desirable to provide gas sensors without significant crosstalk to available gases other than the analysis gas. Furthermore, it may be desirable to provide suitable methods for manufacturing such gas sensors.

### SUMMARY

An aspect of the present disclosure relates to a gas sensor. The gas sensor includes a microelectromechanical systems (MEMS) sensing element, a first cavity arranged in the gas sensor, and a first membrane substantially permeable for particles or molecules of an analysis gas and substantially impermeable for molecules larger than molecules of the analysis gas. The first membrane is configured to allow a diffusion of the analysis gas into the first cavity. The MEMS sensing element is sensitive with respect to the analysis gas diffused into the first cavity.

A further aspect of the present disclosure relates to a method for manufacturing a gas sensor. The method includes generating a MEMS sensing element, generating a first cavity in the gas sensor, and generating a first membrane substantially permeable for molecules of an analysis gas and substantially impermeable for molecules larger than molecules of the analysis gas. The first membrane is configured to allow a diffusion of the analysis gas into the first cavity. The MEMS sensing element is sensitive with respect to the analysis gas diffused into the first cavity.

Those skilled in the art will recognize additional features and advantages upon reading the following detailed description, and upon viewing the accompanying drawings.

### BRIEF DESCRIPTION OF THE DRAWINGS

The present disclosure is illustrated by way of example, and not by way of limitation, in the figures of the accompanying drawings in which like reference numerals refer to similar or identical elements. The elements of the drawings are not necessarily to scale relative to each other. The features of the various illustrated examples can be combined unless they exclude each other.

FIGS. 1A and 1B schematically illustrate a cross-sectional side view and a top view of a gas sensor 100 in accordance with the disclosure.

FIG. 2 schematically illustrates a cross-sectional side view of a gas sensor 200 in accordance with the disclosure.

FIG. 3 schematically illustrates a cross-sectional side view of a gas sensor 300 in accordance with the disclosure.

FIG. 4 schematically illustrates a cross-sectional side view of a gas sensor 400 in accordance with the disclosure.

FIG. 5 schematically illustrates a cross-sectional side view of a gas sensor 500 in accordance with the disclosure.

FIGS. 6A and 6B schematically illustrate a cross-sectional side view and a top view of a gas sensor 600 in accordance with the disclosure.

FIGS. 7A and 7B schematically illustrate a cross-sectional side view and a top view of a membrane 700 which may be included in a gas sensor in accordance with the disclosure.

FIGS. 8A and 8B schematically illustrate a cross-sectional side view and a top view of a membrane 800 which may be included in a gas sensor in accordance with the disclosure.

FIGS. 9A to 9C schematically illustrate a method for manufacturing a membrane 900 which may be included in a gas sensor in accordance with the disclosure.

FIG. 10 illustrates a flowchart of a method for manufacturing a gas sensor in accordance with the disclosure.

### DETAILED DESCRIPTION

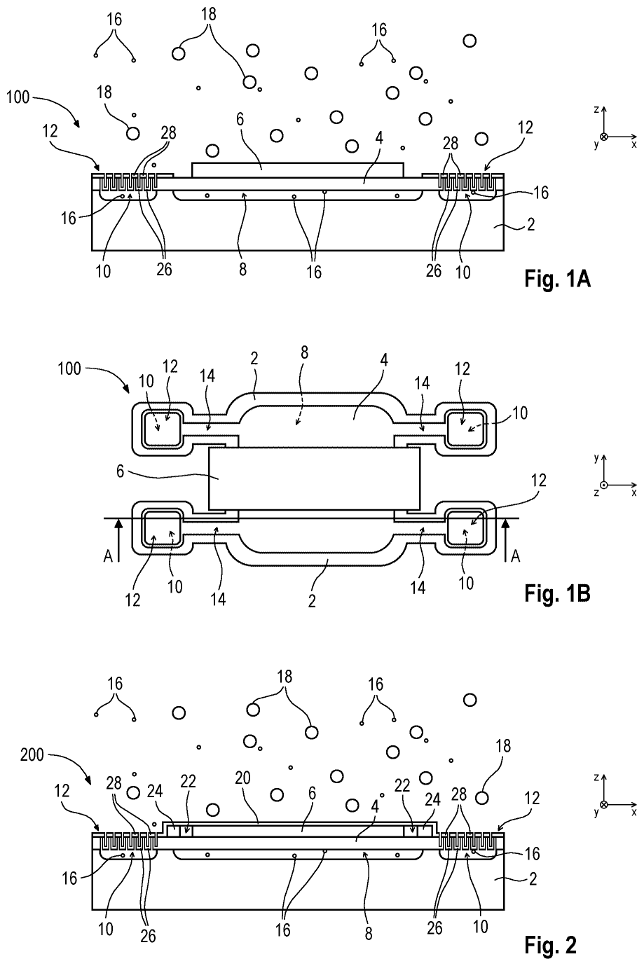

Referring now to FIGS. 1A and 1B, a cross-sectional side view and a top view of a gas sensor 100 in accordance with the disclosure are shown. The cross-sectional side view of FIG. 1A may be along a sectional plane A-A as indicated in the top view of FIG. 1B. The gas sensor 100 may include a semiconductor material 2, a dielectric material 4 arranged above the semiconductor material 2 and a MEMS sensing element 6 arranged above the dielectric material 4. A first cavity 8 and at least one second cavity 10 may be arranged in the semiconductor material 2. In addition, at least one first membrane 12 may be arranged in the dielectric material 4.

The semiconductor material (or semiconductor substrate) 2 may be substantially gastight. In one example, the semiconductor material 2 may include or may correspond to silicon. The semiconductor material 2 may form or may be part of a semiconductor chip (or semiconductor die), such that the gas sensor 100 may also be referred to as gas sensor chip. The first cavity 8 may be at least partially arranged in the semiconductor material 2. In the illustrated example, the first cavity 8 may be limited by the semiconductor material 2 and the dielectric material 4. More particular, the semiconductor material 2 may form a bottom surface and side surfaces of the first cavity 8, while the dielectric material 4 may form a top surface of the first cavity 8. The first cavity 8 may be at least partially arranged beneath the MEMS sensing element 6. More particular, footprints of the first cavity 8 and the MEMS sensing element 6 may at least partially overlap when viewed in the z-direction.

A height of the first cavity 8 measured in the z-direction and a width of the first cavity 8 measured in the x-direction may have a ratio in a range from about 1:10 to about 1:40. For example, the height of the first cavity 8 measured in the z-direction may be in a range from about 1 μm to about 10 μm. In the example top view of FIG. 1B, the first cavity 8 may have a substantially rectangular shape. Here, dimensions of the first cavity 8 measured in the x-direction and the y-direction may be in a range from about 10 μm to about 200 μm, respectively. A typical, but non-limiting value for each dimension of the first cavity 8 measured in the x-direction and the y-direction may be approximately 50 μm.

The second cavities 10 may be at least partially arranged in the semiconductor material 2. In the illustrated example, the gas sensor 100 may include an example and non-limiting number of four second cavities 10. In further examples, the number of second cavities 10 may differ and may be one, two, three or even larger than four. In the shown case, each of the second cavities 10 may be at least partially sealed by a respective first membrane 12. Each of the second cavities 10 may be limited by the semiconductor material 2 and the respective first membrane 12. More particular, the semiconductor material 2 may form a bottom surface and side surfaces of a respective second cavity 10, while the first membrane 12 may form a top surface of the second cavity 10. The second cavities 10 may be connected to the first cavity 8 via at least one channel 14 which may be formed in the semiconductor material 2. The channels 14 may be configured to provide an exchange of an analysis gas between the first cavity 8 and the second cavities 10.

The dielectric material 4 may be made of or may include at least one of an oxide or a nitride, such as silicon oxide. In particular, the dielectric material 4 may be substantially gastight. In the illustrated example, the dielectric material 4 may be formed as a layer covering the first cavity 8 and the second cavities 10. The first membranes 12 may be arranged in the dielectric material 4 at the positions of the second cavities 10.

The first membranes 12 may be substantially permeable for molecules 16 of an analysis gas of interest and substantially impermeable for molecules 18 larger than the molecules 16 of the analysis gas. In one example, the analysis gas may be hydrogen, e.g., the respective first membrane 12 may be substantially permeable for hydrogen. In a further example, the analysis gas may correspond to helium. The other available gas including the larger molecules 18 may include or may correspond to e.g., methane, a hydrocarbon or water (in the gas phase), e.g., the first membrane 12 may be substantially impermeable for at least one of methane molecules, hydrocarbon molecules or water molecules. According to the above, the first membrane 12 may be configured to allow a diffusion of the analysis gas into the second cavities 10 and the first cavity 8, while at the same time the first membrane 12 may be further configured to avoid a diffusion of the other available gas into the second cavities 10 and the first cavity 8.

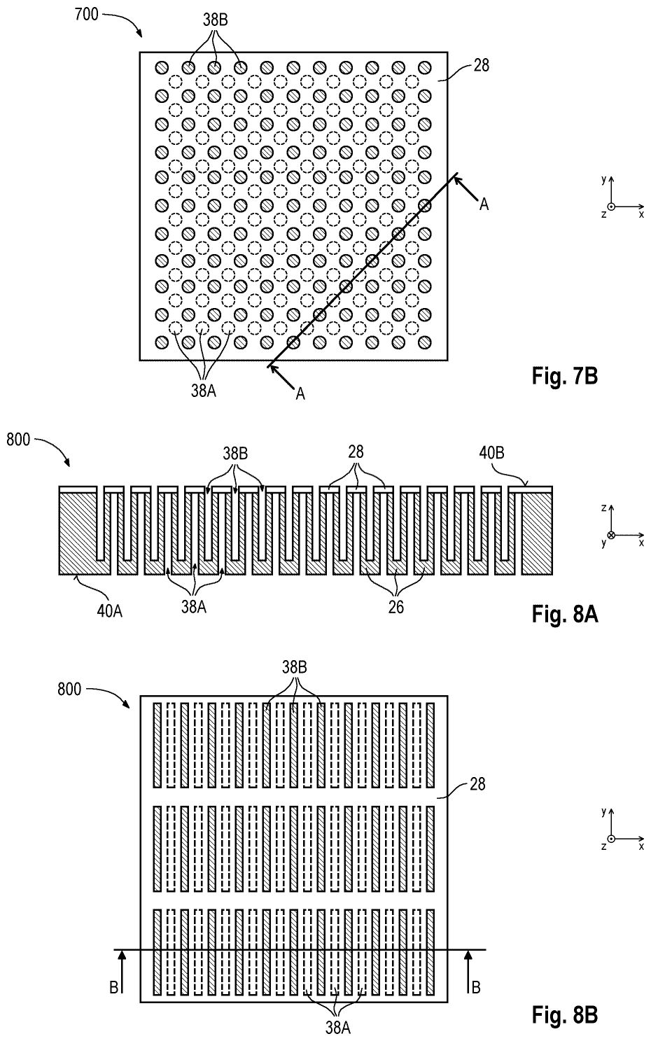

In the illustrated example, the first membrane 12 may be manufactured from a first material 26 and a second material 28. The materials 26 and 28 may be arbitrarily selected as required as long as the first membrane 12 is configured to provide the previously specified permeability features. For example, one or both of the materials 26 and 28 may include or may be made of at least one of thermal oxide, CVD oxide, TEOS, or the like. In one example, the materials 26 and 28 may be similar or the same. In further examples, the materials 26 and 28 may differ. A more detailed description of a membrane which may be included in a gas sensor in accordance with the disclosure and a method for manufacturing thereof is described in connection with FIGS. 7 to 9.

The MEMS sensing element 6 may be sensitive with respect to the analysis gas diffused into the first cavity 8. The MEMS sensing element 6 may be arranged over the top surface of the dielectric material 4 facing away from the first cavity 8. In particular, the MEMS sensing element 6 may be at least partially arranged over the first cavity 8, e.g., footprints of the MEMS sensing element 6 and the first cavity 8 may at least partially overlap when viewed in the z-direction. The MEMS sensing element 6 is not restricted to a specific type or sensing technique. In one example, the MEMS sensing element 6 may be part of or may correspond to a thermal conductivity sensor. In a further example, the MEMS sensing element 6 may be part of or may correspond to a metal oxide semiconductor (MOS) gas sensor. In another example, the MEMS sensing element 6 may be part of or may correspond to a stress sensor which may be configured for an adsorption based analysis gas measurement, such as a palladium film, a cantilever or a membrane. In yet another example, the MEMS sensing element 6 may be based on more than only one of the before mentioned sensing techniques.

The gas sensor 100 may be used for detecting an analysis gas and/or a concentration of the analysis gas in an environment of interest. The molecules 16 of the analysis gas may diffuse through the first membranes 12 and enter the second cavities 10, while larger molecules 18 of other available gases cannot enter the second cavities 10. The molecules 16 diffused into the second cavities 10 may then enter the first cavity 8 through the channels 14 connecting the cavities 8 and 10. In the illustrated example, the first membranes 12 may seal the second cavities 20. However, it is to be understood that in further examples a first membrane 12 may alternatively, or additionally, seal the first cavity 8 such that the analysis gas may directly diffuse into the first cavity 8. In this regard, the second cavities 10 may be regarded as optional. After some time, a concentration of the analysis gas in the first cavity 8 may be similar (or substantially identical) to a concentration of the analysis gas in the environment of the gas sensor 100. The MEMS sensing element 6 being sensitive with respect to the analysis gas diffused into the first cavity 8 may then provide (or output) a measurement signal which may particularly depend on a concentration of the analysis gas in the first cavity 8 and thus a concentration of the analysis in the environment of interest.

The gas sensor 100 may include a first unit (not illustrated) configured to measure the output signal of the MEMS sensing element 6. For example, the first unit may include or may correspond to measurement circuitry configured to measure a voltage or a voltage difference at the MEMS sensing element 6. In addition, the gas sensor 100 may include a second unit (not illustrated) configured to detect the analysis gas and/or a concentration of the analysis gas based on the measured output signal. For example, the second unit may include or may correspond to a logic unit configured to map a measurement value of the measured output signal to a concentration of the analysis gas. The first unit and the second unit may be included in a same single component or in multiple separate components. In one example, at least one of the first unit or the second unit may be integrated in the semiconductor material 2. In a further example, at least one of the first unit or the second unit may be arranged external to the components shown in FIGS. 1A and 1B.

In a specific, but non-limiting example, the MEMS sensing element 6 may include or may correspond to a thermopile which may be arranged on the top surface of the dielectric material 4 at least partially above the first cavity 8. The thermopile may include or may be made of lines of materials having two different Seebeck coefficients in order to generate a thermo-voltage. In addition, a heating element or heater (not illustrated) may be arranged on the top surface of the dielectric material 4 next to the thermopile, wherein the heating element may be configured to generate a temperature gradient across the first cavity 8. For example, the heating element may include or may be made of polysilicon. An analysis gas diffused into the first cavity 8 may form a thermal resistance of the thermopile. The analysis gas contained in the first cavity 8 may act as a thermal bridge. A measured output voltage of the thermopile may depend on the temperature gradient across the first cavity 8 and/or a heat flux through the first cavity 8. As previously described, the first unit may then measure an output voltage of the thermopile and the second unit may detect the analysis gas and/or a concentration of the analysis gas based on the measured output voltage.

In the example of FIGS. 1A and 1B, a measurement performed by the gas sensor 100 may be based on a sum of measuring both a thermal conductivity of the analysis gas contained in the first cavity 8 as well as a thermal conductivity of the gas (e.g., air) above the gas sensor 100. In an example case of the analysis gas being hydrogen, a thermal path extending through the first cavity 8 may have a comparatively low thermal resistance and a comparatively high contribution to the measurement value. The other thermal path extending above the MEMS sensing element 6 may still be cross-sensitive to the other available gas, but may be a smaller contribution to the measurement value.

The gas sensor 100 may outperform conventional gas sensors. Since the first cavity 8 only contains molecules 16 of the analysis gas, but may be substantially free of the larger molecules 18 of other available gases, a cross sensitivity of the gas sensor 100 with respect to gases other than the analysis gas may be suppressed and a measurement accuracy of the gas sensor 100 may be improved compared to conventional gas sensors. In a non-limiting example, a problem of hydrogen sensing solutions for detecting thermal runaway events in lithium ion batteries may be a cross-sensitivity to other gases like methane and other hydrocarbons, but also to humidity. Since the concepts presented herein may lower or even prevent a measurement of such other gases, the cross sensitivities may be removed. No additional measures for tackling cross-sensitivities may thus be required.

Referring now to FIG. 2, a further example of a gas sensor 200 in accordance with the disclosure is shown. The gas sensor 200 may include some or all features of the gas sensor 100 of FIGS. 1A and 1B. The gas sensor 200 may include a second membrane 20 arranged over the MEMS sensing element 6. The second membrane 20 may include or may be made of at least one of thermal oxide, CVD oxide, TEOS, or the like. In one example, the second membrane 20 and the second material 28 of the first membrane 12 may be made of a same material. The dielectric material 4 and the second membrane 20 may form a cavity 22 enclosing the MEMS sensing element 6. More particular, the dielectric material 4 may form a bottom surface of the cavity 22, while the second membrane 20 may form the top surface and side surfaces of the cavity 22. For example, the cavity 22 may be manufactured by utilizing a sacrificial layer (not illustrated) which may first be arranged on the top surface of the dielectric material 4. The second membrane 20 may be formed above the sacrificial layer which may be removed afterwards. The sacrificial layer may include or may be made of carbon in one example. The gas sensor 200 may further include one or more spacer elements 24 which may be arranged on the top surface of the dielectric material 4 and may be configured to provide a constant and reliable distance between the dielectric material 4 and the second membrane 20. For example, the spacer elements 24 may include or may be made of a dielectric material.

The second membrane 20 may be substantially impermeable for molecules 16 of the analysis gas. For example, the second membrane 20 may be substantially hydrogen-tight. A concentration of the analysis gas in the cavity 22 above the dielectric material 4 may thus be smaller than a concentration of the analysis gas in the first cavity 8 beneath the dielectric material 4. Accordingly, a measurement performed by the gas sensor 200 may avoid a measurement contribution of the thermal conductivity of the gas (e.g., air) located above the MEMS sensing element 6 as previously described in connection with the example of FIGS. 1A and 1B. The gas sensor 200 may thus exclusively measure a change in the thermal properties of the first cavity 8. A great selectivity to light gases (such as hydrogen or helium) may thus be provided.

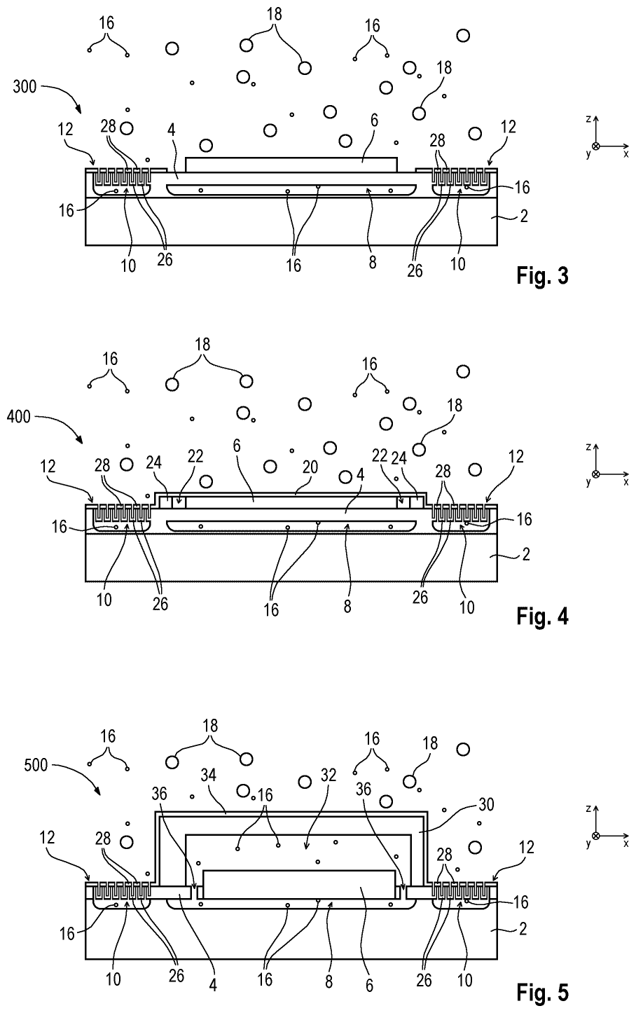

Referring now to FIG. 3, a further example of a gas sensor 300 in accordance with the disclosure is shown. The gas sensor 300 of FIG. 3 may include some or all features of previously described gas sensors. In the illustrated example, the first cavity 8 and the second cavities 10 may be at least partially arranged in the dielectric material 4. In particular, the first cavity 8 may be completely enclosed by the dielectric material 4. That is, the dielectric material 4 may form the bottom surface, the top surface and side surfaces of the first cavity 8. Furthermore, the dielectric material 4 may form the bottom surfaces and the side surfaces of the second cavities 10, while the top surfaces of the second cavities 10 may be formed by the first membranes 12. The first cavity 8 and the second cavities 10 may be connected via channels formed in the dielectric material 4. During an operation of the gas sensor 300, molecules 16 of the analysis gas may diffuse into the second cavities 10 via the first membranes 12 and may then enter the first cavity 8 via the channels formed in the dielectric material 4.

Referring now to FIG. 4, a further example of a gas sensor 400 in accordance with the disclosure is shown. The gas sensor 400 of FIG. 4 may include some or all features of previously described gas sensors. In particular, the gas sensor 400 may combine features of the gas sensors 200 and 300 of FIGS. 2 and 3. Similar to the example of FIG. 2, the gas sensor 400 may include a second membrane 20 arranged over the MEMS sensing element 6. Similar to the example of FIG. 3, the first cavity 8 and the second cavities 10 may be at least partially arranged in the dielectric material 4.

Referring now to FIG. 5, a further example of a gas sensor 500 in accordance with the disclosure is shown. The gas sensor 500 of FIG. 5 may include some or all features of previously described gas sensors. The gas sensor 500 may include a cover (or lid) 30 arranged above the dielectric material 4. In particular, the cover 30 may be mounted on the top surface of the dielectric material 4. The cover 30 may include or may be made of a dielectric material, for example one or more of previously specified dielectric materials. The cover 30 may be gastight and substantially impermeable for molecules 18 larger than molecules 16 of an analysis gas. The MEMS sensing element 6 may be arranged in a third cavity 32 which may be at least partially formed by the dielectric material 4 and the cover 30. The gas sensor 500 may further include a dielectric material 34 which may be arranged on the top surface of the cover 30. In one example, the dielectric material 34 and the second material 28 of the first membrane 12 may be similar or the same.

At least one opening 36 may be formed in the dielectric material 4, wherein the at least one opening 36 may connect the first cavity 8 arranged beneath the MEMS sensing element 6 and the third cavity 32 arranged above the MEMS sensing element 6. During an operation of the gas sensor 500, molecules 16 of the analysis gas may diffuse into the second cavities 10 via the first membranes 12 and may then enter the first cavity 8 via channels formed in e.g., the semiconductor material 2. In addition, the molecules 16 of the analysis gas may enter the third cavity 32 via the at least one opening 36, such that the analysis gas may be located both beneath and above the MEMS sensing element 6.

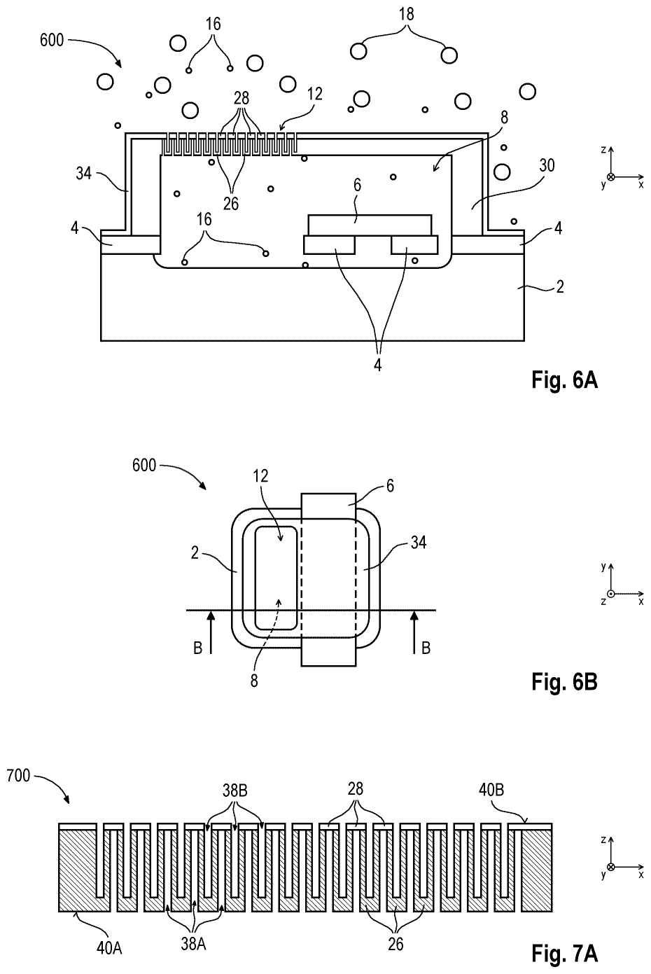

Referring now to FIGS. 6A and 6B, a cross-sectional side view and a top view of a gas sensor 600 in accordance with the disclosure are shown. The cross-sectional side view of FIG. 6A may be along a sectional plane B-B as indicated in the top view of FIG. 6B. The gas sensor 600 may include some or all features of previously described gas sensors. Similar to the example of FIG. 5, the gas sensor 600 may include a cover 30 arranged above the top surface of the dielectric material 4. The gas sensor 600 may include a first cavity 8 in which the MEMS sensing element 6 may be arranged. In the illustrated example, the semiconductor material 2 may form a bottom surface of the first cavity 8, while the cover 30 may form a top surface of the first cavity 8. The side surfaces of the first cavity 8 may be formed by at least one of the cover 30, the dielectric material 4 and the semiconductor material 2. In the shown case, the gas sensor 600 may include a single cavity 8, but not necessarily optional second cavities 10 as described in connection with previous examples.

The gas sensor 600 may include at least one first membrane 12 substantially permeable for molecules 16 of an analysis gas and substantially impermeable for molecules 18 larger than the molecules 16 of the analysis gas. In the illustrated example, the first membrane 12 may be arranged in the cover 30. The molecules 16 of the analysis gas may therefore directly diffuse into the first cavity 8 without any further detour via other cavities.

Referring now to FIGS. 7A and 7B, a cross-sectional side view and a top view of a membrane 700 which may be included in a gas sensor in accordance with the disclosure are shown. The cross-sectional side view of FIG. 7A may be along a sectional plane A-A as indicated in the top view of FIG. 7B. For example, the membrane 700 may correspond to the first membrane 12 in any of the previously described examples. The membrane 700 may include a first portion made of a first material 26 and a second portion made of a second material 28. For example, each of the materials 26 and 28 may include or may be made of at least one of thermal oxide, CVD oxide, TEOS, or the like. In one example, the materials 26 and 28 may be similar or the same. In further examples, the materials 26 and 28 may differ.

The membrane 700 may include a first plurality of first blind holes 38A extending into a first surface 40A of the first membrane 700 and a second plurality of second blind holes 38B extending into a second surface 40B of the first membrane 700 opposite the first surface 40A. Due to a formation of the blind holes 38A and 38B, a thickness of the membrane 700 may be reduced and a low path length through the membrane material may be provided. As a result, the membrane 700 may become permeable for molecules of an analysis gas of interest. For example, a thickness of the membrane 700 may be in a range from about 0.3 μm to about 10 μm. A typical, but non-limiting thickness value may be approximately 1 μm. In the example top view of FIG. 7B, the blind holes 38A and 38B may have a circular shape. In further examples, arbitrary other shapes of the blind holes 38A and 38B may be possible, such as elliptical, quadratic, rectangular, polygonal, or the like. In practice, the first blind holes 38A may not be visible in the top view of FIG. 7B and are indicated by dashed circles. In the cross-sectional side view of FIG. 7A, the membrane 700 may have a meandering shape.

Referring now to FIGS. 8A and 8B, a cross-sectional side view and a top view of a membrane 800 which may be included in a gas sensor in accordance with the disclosure are shown. The cross-sectional side view of FIG. 8A may be along a sectional plane B-B as indicated in the top view of FIG. 8B. The membrane 800 of FIG. 8 may include some or all features of the membrane 700 of FIG. 7. For example, the membrane 800 may correspond to the first membrane 12 in any of the previously described examples. In the example top view of FIG. 8B, the blind holes 38A and 38B may have a rectangular shape.

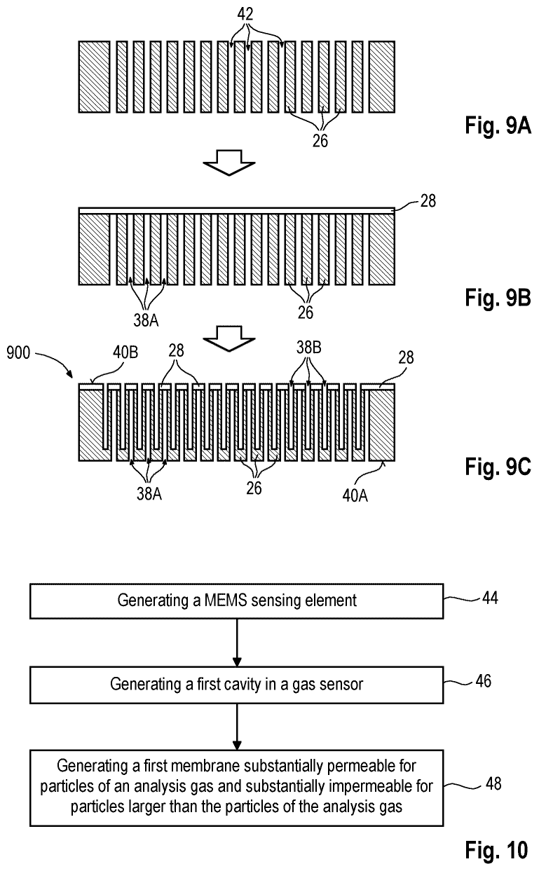

Referring now to FIGS. 9A to 9C, a method for manufacturing a membrane 900 which may be included in a gas sensor in accordance with the disclosure is shown. For example, the membranes 700 and 800 of FIGS. 7 and 8 or the first membranes 12 of previous examples may be manufactured based on the method of FIGS. 9A to 9C.

In FIG. 9A, a first material 26 may be provided. For example, the first material 26 may include or may be made of at least one of thermal oxide, CVD oxide, TEOS, or the like. The first material 26 may be perforated, wherein a plurality of through holes 42 may be formed in the first material 26. Any suitable technique may be used for manufacturing the through holes 42. In one example, an etching process may be performed, wherein an etch stop material and/or an etch mask may be used accordingly. The through holes 42 may extend from the top surface of the first material 26 to the bottom surface of the first material 26 in a substantially vertical direction.

In FIG. 9B, a second material 28 may be arranged above the top surface of the first material 26, wherein the openings of the through holes 42 in the top surface of the first material 26 may be covered by the second material 28 and a plurality of first blind holes 38A may be formed. A thickness of the second material 28 measured in the z-direction may be similar or equal to a dimension of a through hole 42 measured in the x-direction. For example, the second material 28 may include or may be made of at least one of thermal oxide, CVD oxide, TEOS, or the like.

In FIG. 9C, a plurality of second blind holes 38B may be formed in the top surface of the first material 26 laterally offset to the first blind holes 38A. For example, the second blind holes 38B may be manufactured by etching the top surface of the first material 26. In this regard, an etch stop material and/or an etch mask may be used accordingly.

FIG. 10 illustrates a flowchart of a method for manufacturing a gas sensor in accordance with the disclosure. The method is described in a general manner to qualitatively specify aspects of the disclosure. The method may include further aspects and may be extended by any of the aspects described in connection with other examples. For example, any of the previously described gas sensors may be manufactured based on the method of FIG. 10.

In a step 44, a MEMS sensing element may be generated. In a step 46, a first cavity may be generated in the gas sensor. In a step 48, a first membrane substantially permeable for molecules of an analysis gas and substantially impermeable for molecules larger than molecules of the analysis gas may be generated. The first membrane may be configured to allow a diffusion of the analysis gas into the first cavity. The MEMS sensing element may be sensitive with respect to the analysis gas diffused into the first cavity.

Gas sensors in accordance with the disclosure may particularly be used as hydrogen sensors for detecting hydrogen and/or hydrogen concentrations. Hydrogen sensors may be used in a variety of applications, such as in the automotive sector or industrial applications. By way of example, hydrogen sensors may be used for hydrogen exhaust gas detection, exhaust gas monitoring, battery monitoring, battery management, hydrogen sensing, hydrogen leakage detection, hydrogen detection in industrial plants, etc.

With a view to achieving climate targets, the automotive industry is promoting and developing the production of hydrogen-powered vehicles. Fuel cell cars can be considered as a breakthrough for electromobility and can heavily contribute to a reduced CO₂ emission. Gas sensors as described herein improve hydrogen technology and may thus contribute to achieving climate targets that have been set. Improved gas sensors in accordance with the disclosure and methods for operating such sensors may contribute to green technology and green power solutions, e.g., climate-friendly solutions providing reduced energy usage.

### ASPECTS

In the following, gas sensors and methods for manufacturing gas sensors are explained using aspects.

Aspect 1 is a gas sensor, comprising: a MEMS sensing element; a first cavity arranged in the gas sensor; and a first membrane substantially permeable for molecules of an analysis gas and substantially impermeable for molecules larger than molecules of the analysis gas, wherein the first membrane is configured to allow a diffusion of the analysis gas into the first cavity, and wherein the MEMS sensing element is sensitive with respect to the analysis gas diffused into the first cavity.

Aspect 2 is a gas sensor according to Aspect 1, further comprising: at least one second cavity connected to the first cavity, wherein the at least one second cavity is at least partially sealed by the first membrane.

Aspect 3 is a gas sensor according to Aspect 1 or 2, further comprising: a semiconductor material; and a dielectric material arranged over the semiconductor material, wherein the first membrane is arranged in the dielectric material.

Aspect 4 is a gas sensor according to Aspect 3, wherein the first cavity is at least partially arranged in the semiconductor material.

Aspect 5 is a gas sensor according to Aspect 3 or 4, wherein: the at least one second cavity is arranged in the semiconductor material, and the first cavity and the at least one second cavity are connected via at least one channel formed in the semiconductor material.

Aspect 6 is a gas sensor according to any of Aspects 3 to 5, wherein the first cavity is at least partially arranged in the dielectric material.

Aspect 7 is a gas sensor according to any of Aspects 3 to 6, wherein: the at least one second cavity is arranged in the dielectric material, and the first cavity and the at least one second cavity are connected via at least one channel formed in the dielectric material.

Aspect 8 is a gas sensor according to any of Aspects 3 to 7, wherein the MEMS sensing element is arranged over a surface of the dielectric material facing away from the first cavity.

Aspect 9 is a gas sensor according to any of the preceding Aspects, wherein a height of the first cavity and a width of the first cavity have a ratio in a range from 1:10 to 1:40.

Aspect 10 is a gas sensor according to any of the preceding Aspects, wherein a height of the first cavity is in a range from 1 μm to 10 μm.

Aspect 11 is a gas sensor according to any of the preceding Aspects, further comprising: a second membrane arranged over the MEMS sensing element, wherein the second membrane is substantially impermeable for molecules of the analysis gas.

Aspect 12 is a gas sensor according to any of the preceding Aspects, further comprising: a cover arranged over the dielectric material, wherein the MEMS sensing element is arranged in a third cavity at least partially formed by the dielectric material and the cover.

Aspect 13 is a gas sensor according to Aspect 12, further comprising: at least one opening formed in the dielectric material, wherein the at least one opening connects the first cavity and the third cavity.

Aspect 14 is a gas sensor according to Aspect 12 or 13, wherein the first membrane is arranged in the cover.

Aspect 15 is a gas sensor according to any of the preceding Aspects, wherein the MEMS sensing element comprises at least one of a thermal conductivity sensor, a MOS gas sensor or a stress sensor configured for an adsorption based analysis gas measurement.

Aspect 16 is a gas sensor according to any of the preceding Aspects, wherein: the MEMS sensing element comprises a thermopile, and the analysis gas diffused into the first cavity forms a thermal resistance of the thermopile.

Aspect 17 is a gas sensor according to any of the preceding Aspects, wherein the first membrane comprises a first plurality of first blind holes extending into a first surface of the first membrane and a second plurality of second blind holes extending into a second surface of the first membrane opposite the first surface.

Aspect 18 is a gas sensor according to any of the preceding Aspects, wherein the first membrane is substantially permeable for hydrogen.

Aspect 19 is a gas sensor according to any of the preceding Aspects, wherein the first membrane is substantially impermeable for at least one of methane molecules, hydrocarbon molecules or water molecules.

Aspect 20 is a gas sensor according to any of the preceding Aspects, further comprising: a first unit configured to measure an output signal of the MEMS sensing element; and a second unit configured to detect at least one of the analysis gas or a concentration of the analysis gas based on the measured output signal.

Aspect 21 is a method for manufacturing a gas sensor, the method comprising: generating a MEMS sensing element; generating a first cavity in the gas sensor; and generating a first membrane substantially permeable for molecules of an analysis gas and substantially impermeable for molecules larger than molecules of the analysis gas, wherein the first membrane is configured to allow a diffusion of the analysis gas into the first cavity, and wherein the MEMS sensing element is sensitive with respect to the analysis gas diffused into the first cavity.

Although specific aspects have been illustrated and described herein, it will be appreciated by those of ordinary skill in the art that a variety of alternate and/or equivalent implementations may be substituted for the specific aspects shown and described without departing from the scope of the present implementation. This application is intended to cover any adaptations or variations of the specific aspects discussed herein. Therefore, it is intended that this implementation be limited only by the claims and the equivalents thereof.

It should be noted that the methods and devices including its preferred implementations as outlined in the present document may be used stand-alone or in combination with the other methods and devices disclosed in this document. In addition, the features outlined in the context of a device are also applicable to a corresponding method, and vice versa. Furthermore, all aspects of the methods and devices outlined in the present document may be arbitrarily combined. In particular, the features of the claims may be combined with one another in an arbitrary manner.

It should be noted that the description and drawings merely illustrate the principles of the proposed methods and systems. Those skilled in the art will be able to implement various arrangements that, although not explicitly described or shown herein, embody the principles of the implementation and are included within its spirit and scope. Furthermore, all aspects and implementations outlined in the present document are principally intended expressly to be only for explanatory purposes to help the reader in understanding the principles of the proposed methods and systems. Furthermore, all statements herein providing principles, aspects, and implementations of the implementation, as well as specific aspects thereof, are intended to encompass equivalents thereof.

1. A gas sensor, comprising:
a microelectromechanical systems (MEMS) sensing element;
a first cavity arranged in the gas sensor; and
a first membrane substantially permeable for molecules of an analysis gas and substantially impermeable for molecules larger than molecules of the analysis gas,
wherein the first membrane is configured to allow a diffusion of the analysis gas into the first cavity, and
wherein the MEMS sensing element is sensitive with respect to the analysis gas diffused into the first cavity.

2. The gas sensor of claim 1, further comprising:
at least one second cavity connected to the first cavity, wherein the at least one second cavity is at least partially sealed by the first membrane.

3. The gas sensor of claim 2, further comprising:
a semiconductor material; and
a dielectric material arranged over the semiconductor material, wherein the first membrane is arranged in the dielectric material.

4. The gas sensor of claim 3, wherein the first cavity is at least partially arranged in the semiconductor material.

5. The gas sensor of claim 3, wherein:
the at least one second cavity is arranged in the semiconductor material, and
the first cavity and the at least one second cavity are connected via at least one channel formed in the semiconductor material.

6. The gas sensor of claim 3, wherein the first cavity is at least partially arranged in the dielectric material.

7. The gas sensor of claim 3, wherein:
the at least one second cavity is arranged in the dielectric material, and
the first cavity and the at least one second cavity are connected via at least one channel formed in the dielectric material.

8. The gas sensor of claim 3, wherein the MEMS sensing element is arranged over a surface of the dielectric material facing away from the first cavity.

9. The gas sensor of claim 1, wherein a height of the first cavity and a width of the first cavity have a ratio in a range from 1:10 to 1:40.

10. The gas sensor of claim 1, wherein a height of the first cavity is in a range from 1 μm to 10 μm.

11. The gas sensor of claim 1, further comprising:
a second membrane arranged over the MEMS sensing element, wherein the second membrane is substantially impermeable for molecules of the analysis gas.

12. The gas sensor of claim 3, further comprising:
a cover arranged over the dielectric material, wherein the MEMS sensing element is arranged in a third cavity at least partially formed by the dielectric material and the cover.

13. The gas sensor of claim 12, further comprising:
at least one opening formed in the dielectric material, wherein the at least one opening connects the first cavity and the third cavity.

14. The gas sensor of claim 12, wherein the first membrane is arranged in the cover.

15. The gas sensor of claim 1, wherein the MEMS sensing element comprises at least one of a thermal conductivity sensor, a MOS gas sensor or a stress sensor configured for an adsorption based analysis gas measurement.

16. The gas sensor of claim 1, wherein:
the MEMS sensing element comprises a thermopile, and
the analysis gas diffused into the first cavity forms a thermal resistance of the thermopile.

17. The gas sensor of claim 1, wherein the first membrane comprises a first plurality of first blind holes extending into a first surface of the first membrane and a second plurality of second blind holes extending into a second surface of the first membrane opposite the first surface.

18. The gas sensor of claim 1, wherein the first membrane is substantially permeable for hydrogen.

19. The gas sensor of claim 1, wherein the first membrane is substantially impermeable for at least one of methane molecules, hydrocarbon molecules or water molecules.

20. The gas sensor of claim 1, further comprising:
a first unit configured to measure an output signal of the MEMS sensing element; and
a second unit configured to detect at least one of the analysis gas or a concentration of the analysis gas based on the measured output signal.

21. A method for manufacturing a gas sensor, the method comprising:
generating a microelectromechanical systems (MEMS) sensing element;
generating a first cavity in the gas sensor; and
generating a first membrane substantially permeable for molecules of an analysis gas and substantially impermeable for molecules larger than molecules of the analysis gas,
wherein the first membrane is configured to allow a diffusion of the analysis gas into the first cavity, and
wherein the MEMS sensing element is sensitive with respect to the analysis gas diffused into the first cavity.
